(19) United States
(12) Patent Application Publication
(10) Pub. No.: US 2023/0356104 A1
(43) Pub. Date: Nov. 9, 2023

(54) CRYSTALLIZATION SYSTEM AND METHOD USING THERMAL TREATMENT

(71) Applicant: DHF AMERICA LLC, Raleigh, NC (US)

(72) Inventors: Tomoaki SAKURAI, Tokyo (JP); Isamu UMEDA, Tokyo (JP); Kotaro HIRAYAMA, Tokyo (JP); Kyoko HIRAYAMA, Tokyo (JP)

(21) Appl. No.: 18/040,348

(22) PCT Filed: Aug. 2, 2021
(86) PCT No.: PCT/US2021/044161

§ 371 Date: Feb. 2, 2023

Related U.S. Application Data
(60) Provisional application No. 63/061,529, filed on Aug. 5, 2020.
(60) Provisional application No. 63/154,933, filed on Mar. 1, 2021.

(51) Int. Cl. B01D 9/00; C07C 51/43

(52) U.S. Cl. CPC B01D 9/0031; B01D 9/0063; C07C 51/43

(57) ABSTRACT

A method of crystallizing a solution (24) having at least one compound mixed (18) with a dissolving agent (20) is provided. The method includes performing a heating process by heating the solution (24) until a current temperature of the solution is equal to a predetermined treatment temperature, maintaining the current temperature at the predetermined treatment temperature for a predetermined treatment time period, performing a cooling process by cooling the solution (24) until the current temperature is less than the predetermined treatment temperature and a crystallization temperature of the at least one compound (18), causing formation of a plurality of crystal particles (30) of the at least one compound by cooling the solution until the current temperature is equal to a predetermined termination temperature of the at least one compound (18), and varying a particle size of each of the crystal particles (30) based on a cooling speed of the solution.

### CROSS-REFERENCE TO RELATED APPLICATIONS

The present application claims priority to co-pending U.S. Provisional Application No. 63/061,529, filed Aug. 5, 2020, titled “CRYSTALLIZATION SYSTEM AND METHOD USING THERMAL TREATMENT,” and U.S. Provisional Application No. 63/154,933, filed Mar. 1, 2021, titled “CRYSTALLIZATION SYSTEM AND METHOD USING THERMAL TREATMENT,” which are incorporated by reference herein in their entireties.

### BACKGROUND

The present invention relates to a crystallization process for purifying a compound, and more specifically relates to a crystallization system and method using thermal treatment for purifying the compound using specific heating and cooling techniques.

Crystallization processes are used to recycle a compound mixed in polyethylene terephthalate (PET) products for reducing impurities in the compound. An exemplary compound includes terephthalic acid (TPA) typically used for PET production or to provide various characteristics as a raw material in the PET products.

Recently, due to fast population growth and economic development, the utilization of TPA has increased at an alarming rate despite the effort of continuous recycling movement to alleviate the natural environmental pollution. To correspond with an increased need in TPA production, TPA is recycled after use using the crystallization process.

For example, crude TPA can be generated by bromine-promoted catalytic oxidation of p-xylene and purified by a hydrogenation process. During purification, an existing crystallization process often uses a large pressurized reactor to produce purified terephthalic acid (PTA). However, for the large pressurized reactor to provide an adequate reactivity during the crystallization process, a reaction field associated with the large pressurized reactor needs to be volumetrically large, causing the reaction field to be slow to adapt and difficult to be controlled during the crystallization process.

As such, the large pressurized reactor is expensive and regulated by government due to a reactor size. Further, controlling heating/cooling operation is time-consuming and complicated when using the existing crystallization process. Consequently, PTA is produced at significantly high capital and operating costs.

Moreover, during the cooling operation of crystallization, a particle size of each crystal particle is an important factor in recyclability of PTA because a characteristic of the particle size determines useful physical or chemical properties of PTA. However, the existing crystallization process is slow and uses an uneconomic and laborious control system and method to determine the particle size during the cooling operation of the reactor.

Such undesirable usage of the large pressurized reactor and heating/cooling operation makes it difficult to produce PTA at a reasonable time and expense. To produce PTA having a desired particle size, an effective crystallization of TPA may not be achieved without precisely controlling the heating/cooling operation.

Thus, there is a need to develop enhanced crystallization techniques and equipment that overcome one or more above-described disadvantages of the existing crystallization process.

### SUMMARY

In one embodiment of the present disclosure, a method of crystallizing a solution having at least one compound mixed with a dissolving agent is provided. The method includes performing a heating process for the solution by heating the solution until a current temperature of the solution is equal to a predetermined treatment temperature, maintaining the current temperature of the solution at the predetermined treatment temperature for a predetermined treatment time period, performing a cooling process for the solution by cooling the solution until the current temperature of the solution is less than the predetermined treatment temperature and a crystallization temperature of the at least one compound, causing formation of a plurality of crystal particles of the at least one compound in the solution by cooling the solution until the current temperature of the solution is equal to a predetermined termination temperature of the at least one compound, and varying a particle size of each of the plurality of crystal particles based on a cooling speed of the solution during the cooling process utilizing the cooling process having a plurality of cooling phases.

In one example, the method includes including a first phase and a second phase in the plurality of cooling phases. In a variation, the method includes decreasing the current temperature of the solution from the predetermined treatment temperature to the crystallization temperature of the at least one compound during the first phase. In a further variation, the method includes decreasing the current temperature of the solution from the crystallization temperature of the at least one compound to the predetermined termination temperature of the at least one compound during the second phase.

In another example, the method includes storing the solution in a thermal treatment vessel having a configuration defined by a ratio of an inner volume and at least one of: an inner surface area of the thermal treatment vessel and an outer surface area of the thermal treatment vessel. In a variation, the method includes defining the ratio by a value of the outer surface area of the thermal treatment vessel divided by the inner volume of the thermal treatment vessel.

In yet another example, the method includes determining a cooling speed associated with crystallizing the solution based on a desired crystal size of the at least one compound. In a variation, the method includes selecting a cooling method used for crystallizing the solution based on the determined cooling speed. In another variation, the method includes performing the cooling process for the solution by cooling the solution using the selected cooling method and the determined cooling speed. In yet another variation, the method includes satisfying a predetermined variation allowance of the plurality of crystal particles of the at least one compound in the solution in relation to the desired crystal size of the at least one compound based on the selected cooling method and the determined cooling speed. In still another variation, the method includes satisfying a predetermined variation allowance of the plurality of crystal particles of the at least one compound in the solution in relation to the desired crystal size of the at least one compound based on a ratio associated with a reaction field of a thermal treatment vessel used in the cooling process.

In another embodiment of the present disclosure, a system of crystallizing a solution having at least one compound mixed with a dissolving agent is provided. The system includes a heating assembly configured to perform a heating process for the solution by heating the solution until a current temperature of the solution is equal to a predetermined treatment temperature. The heating assembly is configured to maintain the current temperature of the solution at the predetermined treatment temperature for a predetermined treatment time period. Also included in the system is a cooling device configured to perform a cooling process for the solution by cooling the solution until the current temperature of the solution is less than the predetermined treatment temperature and a crystallization temperature of the at least one compound. A controller is configured to: cause formation of a plurality of crystal particles of the at least one compound in the solution by cooling the solution until the current temperature of the solution is equal to a predetermined termination temperature of the at least one compound, and vary a particle size of each of the plurality of crystal particles based on a cooling speed of the solution during the cooling process utilizing the cooling process having a plurality of cooling phases.

In one example, the plurality of cooling phases includes a first phase and a second phase. In a variation, the controller is configured to decrease the current temperature of the solution from the predetermined treatment temperature to the crystallization temperature of the at least one compound during the first phase. In another variation, the controller is configured to decrease the current temperature of the solution from the crystallization temperature of the at least one compound to the predetermined termination temperature of the at least one compound during the second phase.

In another example, the system further includes a thermal treatment vessel configured to store the solution in the thermal treatment vessel having a configuration defined by a ratio of an inner volume and at least one of: an inner surface area of the thermal treatment vessel and an outer surface area of the thermal treatment vessel. In a variation, the ratio is defined by a value of the outer surface area of the thermal treatment vessel divided by the inner volume of the thermal treatment vessel.

In yet another example, the controller is configured to determine a cooling speed associated with crystallizing the solution based on a desired crystal size of the at least one compound. In a variation, the controller is configured to select a cooling method used for crystallizing the solution based on the determined cooling speed. In another variation, the controller is configured to perform the cooling process for the solution by cooling the solution using the selected cooling method and the determined cooling speed. In yet another variation, the controller is configured to satisfy a predetermined variation allowance of the plurality of crystal particles of the at least one compound in the solution in relation to the desired crystal size of the at least one compound based on the selected cooling method and the determined cooling speed. In still another variation, the controller is configured to satisfy a predetermined variation allowance of the plurality of crystal particles of the at least one compound in the solution in relation to the desired crystal size of the at least one compound based on a ratio associated with a reaction field of a thermal treatment vessel used in the cooling process.

In yet another embodiment of the present disclosure, a method of crystallizing a solution having at least one compound mixed with a dissolving agent is provided. The method includes performing a heating process for the solution by heating the solution until a current temperature of the solution is equal to a predetermined treatment temperature, maintaining the current temperature of the solution at the predetermined treatment temperature for a predetermined treatment time period, determining a cooling speed associated with crystallizing the solution based on a desired crystal size of the at least one compound, selecting a cooling method used for crystallizing the solution based on the determined cooling speed, performing a cooling process for the solution by cooling the solution using the selected cooling method and the determined cooling speed, causing formation of a plurality of crystal particles of the at least one compound in the solution by cooling the solution until the current temperature of the solution is equal to a predetermined termination temperature of the at least one compound, and satisfying a predetermined variation allowance of the plurality of crystal particles of the at least one compound in the solution in relation to the desired crystal size of the at least one compound based on the selected cooling method and the determined cooling speed.

In still another embodiment of the present disclosure, a system of crystallizing a solution having at least one compound mixed with a dissolving agent is provided. The system includes a heating assembly configured to perform a heating process for the solution by heating the solution until a current temperature of the solution is equal to a predetermined treatment temperature. The heating assembly is configured to maintain the current temperature of the solution at the predetermined treatment temperature for a predetermined treatment time period. A cooling device is configured to perform a cooling process for the solution by cooling the solution until the current temperature of the solution is less than the predetermined treatment temperature and a crystallization temperature of the at least one compound. The cooling device is configured to: determine a cooling speed associated with crystallizing the solution based on a desired crystal size of the at least one compound, select a cooling method used for crystallizing the solution based on the determined cooling speed, perform the cooling process for the solution using the selected cooling method and the determined cooling speed, cause formation of a plurality of crystal particles of the at least one compound in the solution by cooling the solution until the current temperature of the solution is equal to a predetermined termination temperature of the at least one compound, and satisfy a predetermined variation allowance of the plurality of crystal particles of the at least one compound in the solution in relation to the desired crystal size of the at least one compound based on the selected cooling method and the determined cooling speed.

The methods, systems, and apparatuses disclosed herein may be implemented in any means for achieving various aspects. Other features will be apparent from the accompanying drawings and from the detailed description that follows.

### BRIEF DESCRIPTION OF THE DRAWINGS

Example embodiments are illustrated by way of example and not limitation in the figures of the accompanying drawings, in which like references indicate similar elements and in which:

FIG. 1 illustrates a schematic diagram of an exemplary crystallization system featuring a thermal treatment vessel in accordance with embodiments of the present disclosure;

FIG. 2 illustrates a schematic diagram of another exemplary configuration of the thermal treatment vessel used in the crystallization system of FIG. 1;

FIG. 3 illustrates a schematic diagram of an exemplary arrangement of the thermal treatment vessel used in the crystallization system of FIG. 1;

FIG. 4 illustrates a schematic diagram of another exemplary arrangement of the thermal treatment vessel used in the crystallization system of FIG. 1;

FIG. 5 is a flow chart of an exemplary crystallization method using thermal treatment when operating the crystallization system shown in FIGS. 1-4 in accordance with embodiments of the present disclosure;

FIG. 6 illustrates an exemplary graphical presentation of a relationship between a current temperature and a time period during a heating operation of the crystallization system of FIG. 1;

FIG. 7 illustrates an exemplary graphical presentation of a relationship between the current temperature and the time period during the heating operation of the crystallization system of FIG. 2;

FIG. 8 illustrates exemplary graphical presentations of a relationship between the current temperature and a particle size during a cooling operation of the crystallization system of FIG. 1;

FIG. 9 illustrates other exemplary graphical presentations of a relationship between the current temperature and the particle size during the cooling operation of the crystallization system of FIG. 1;

FIG. 10 illustrates a schematic diagram of another exemplary crystallization system featuring an inline thermal treatment vessel in accordance with embodiments of the present disclosure;

FIG. 11 illustrates an exemplary backpressure regulator used with the crystallization system of FIG. 10;

FIG. 12 illustrates another exemplary backpressure regulator used with the crystallization system of FIG. 10;

FIG. 13 is a flow chart of another exemplary crystallization method using thermal treatment when operating the crystallization system shown in FIGS. 10-12 in accordance with embodiments of the present disclosure;

FIG. 14 illustrates an exemplary graphical presentation of a relationship between a cooling speed and a particle size during a cooling operation performed by the crystallization system of FIG. 10; and

FIG. 15 illustrates an exemplary graphical presentation of a relationship between a number of crystal particles and the particle size during the cooling operation performed by the crystallization system of FIG. 10.

Other features of the present embodiments will be apparent from the accompanying drawings and from the detailed description that follows.

### DETAILED DESCRIPTION

Embodiments of the present disclosure will be described in detail herebelow with reference to the attached drawings.

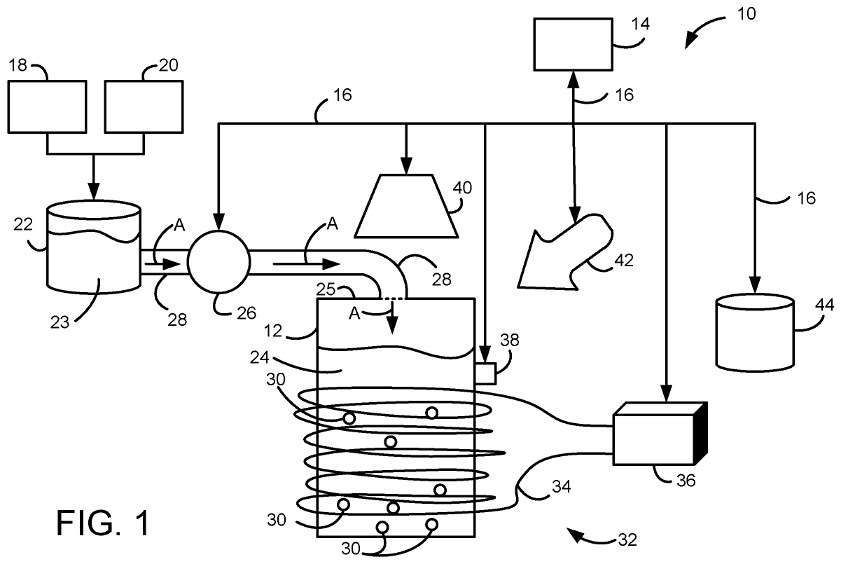

Referring now to FIG. 1, an exemplary crystallization system 10 having a thermal treatment vessel 12 is shown in accordance with embodiments of the present disclosure. In the illustrated embodiment, the crystallization system 10 includes a control system 14 communicably connected to various components associated with the thermal treatment vessel 12 via a network 16, and configured to instruct the various components for operation of a crystallization process.

During the crystallization process, a compound 18 is mixed with a dissolving agent 20 in a mixer 22 to form a sludge 23, such as a slurry. In one embodiment, the compound 18 is a recyclable material, such as crude TPA, and the dissolving agent 20 is a liquid solvent, such as water. An exemplary ratio between the compound 18 and the dissolving agent 20 is approximately 1:5 (20% of crude TPA and 80% of water). In one embodiment, the mixer 22 is operated by the control system 14 such that crude TPA is incorporated into water for creating the sludge 23.

In one embodiment, the control system 14 instructs a pump 26 to retrieve the sludge 23 from the mixer 22 via a conduit 28 in a flow direction designated by an arrow A. Retrieved sludge 23 is then delivered to the thermal treatment vessel 12 via the conduit 28 for the crystallization process.

Upon completion of the delivery of the sludge 23, the thermal treatment vessel 12 is sealed airtight using a cover 25, such as a screw cap. In embodiments, the sludge 23 is heated in the thermal treatment vessel 12 until the compound 18 is melted in the sludge 23, and at such time, the sludge 23 becomes a solution 24. In operation, the crystallization system 10 performs the crystallization process using thermal treatment for generating a plurality of crystal particles 30 associated with the compound 18.

In one embodiment, the thermal treatment vessel 12 is configured for securely storing a proper amount of the solution 24 and also configured to be heated by a heating assembly 32 for functioning as a heating kiln body. In one example, the heating assembly 32 can be an induction heating system. In another example, the heating assembly 32 can be any suitable heating device, such as an electric, gas, or oil heater to suit different applications.

An exemplary configuration of the thermal treatment vessel 12 has a reservoir capacity of approximately 50-100 milliliters (ml), which is much smaller than the existing pressurized reactor typically used for the crystallization process, and an inner pressure capability of approximately 4-7 megapascal (MPa). However, the reservoir capacity and the inner pressure capability can vary to suit different applications.

In certain embodiments, the thermal treatment vessel 12 has a configuration defined by a ratio of an inner volume and an outer surface area of the thermal treatment vessel 12. In one embodiment, the outer surface area of the thermal treatment vessel 12 refers to an outer skin surface area directly contoured to complement an inner surface area of the thermal treatment vessel 12. In another embodiment, the outer surface area of the thermal treatment vessel 12 does not vary depending on a configuration of the outer surface area, but is consistently commensurate with the inner surface area of the thermal treatment vessel 12. To sustain an overall size of the thermal treatment vessel 12 relatively small, the ratio of the inner volume and the outer surface area is maintained at a predetermined value. In this configuration, the reaction field in the inner volume of the thermal treatment vessel 12 can be volumetrically small and thus becomes easy to be controlled and fast to adapt to the crystallization process.

An exemplary ratio associated with the reaction field between the inner volume and the outer surface area of the thermal treatment vessel 12 is shown below in expression (1):

$$R = \frac{{A}_{OUT}}{V} \qquad ( 1 )$$

wherein R denotes a specific surface ratio between an inner volume V (e.g., cubic millimeter or mm³) of the thermal treatment vessel 12 and an outer surface area A_{OUT} (e.g., square millimeter or mm²) of the thermal treatment vessel 12. In one embodiment, the ratio R is greater than or equal to 0.125 mm⁻¹, and an exemplary predetermined value of the ratio R is approximately 0.13 (e.g., mm⁻¹). Although a unit of millimeter is used in this example, any suitable unit, such as meter or inch, is also contemplated to suit the application.

In some embodiments, the thermal treatment vessel 12 has a configuration defined by a ratio of an inner volume and an inner surface area of the thermal treatment vessel 12. In one embodiment, the inner surface area of the thermal treatment vessel 12 refers to an inner skin surface area directly contoured to complement an inner surface area of the thermal treatment vessel 12. In another embodiment, the inner surface area of the thermal treatment vessel 12 does not vary depending on a configuration of the inner surface area, but is consistently commensurate with the outer surface area of the thermal treatment vessel 12. To sustain an overall size of the thermal treatment vessel 12 relatively small, the ratio of the inner volume and the inner surface area is maintained at a predetermined value. In this configuration, the reaction field in the inner volume of the thermal treatment vessel 12 can be volumetrically small and thus becomes easy to be controlled and fast to adapt to the crystallization process.

Another exemplary ratio associated with the reaction field between the inner volume and the inner surface area of the thermal treatment vessel 12 is shown below in expression (2):

$$R = \frac{{A}_{IN}}{V} \qquad ( 2 )$$

wherein R denotes a specific surface ratio between an inner volume V (e.g., cubic millimeter or mm³) of the thermal treatment vessel 12 and an inner surface area A_{IN} (e.g., square millimeter or mm²) of the thermal treatment vessel 12. In one embodiment, the ratio R is greater than or equal to 0.125 mm⁻¹, and an exemplary predetermined value of the ratio R is approximately 0.13 (e.g., mm⁻¹). Although a unit of millimeter is used in this example, any suitable unit, such as meter or inch, is also contemplated to suit the application.

To perform the crystallization process, the crystallization system 10 performs a heating process and a cooling process wherein the cooling process occurs after completion of the heating process. In one embodiment, before the heating process, the thermal treatment vessel 12 having the sludge 23 is sealed airtight by closing the cover 25. To initiate the heating process, the heating assembly 32 inductively heats the thermal treatment vessel 12 to a predetermined treatment temperature for a predetermined treatment time period while the sludge 23 is securely stored in the thermal treatment vessel 12.

In one embodiment, the predetermined treatment temperature is higher than a melting temperature of the compound 18 so that the compound 18 can be melted in the dissolving agent 20. After the sludge 23 is heated for the predetermined treatment time period, the sludge 23 becomes the solution 24.

An exemplary treatment temperature ranges approximately between 100-750 degrees Celsius (° C.), and an exemplary treatment time period ranges approximately between 1-60 minutes. However, the treatment temperature range and the treatment time period range can vary to suit different applications. In one embodiment, the treatment temperature ranges approximately between 100-400° C., and in another embodiment, the treatment temperature ranges approximately between 100-300° C. For example, the treatment temperature is approximately 345° C.

In one embodiment, during the thermal treatment induced by the heating assembly 32, the thermal treatment vessel 12 can reach the predetermined treatment temperature of approximately 270° C. and a predetermined inner pressure of approximately 5.5 MPa. In this case, the crystallization system 10 performs a subcritical water treatment for the crystallization process of TPA.

In one embodiment, the heating assembly 32 includes one or more heating induction coils 34 configured to surround at least a portion of the thermal treatment vessel 12, and a high-frequency power supply unit 36 configured to inductively drive the heating induction coils 34. In one embodiment, the thermal treatment vessel 12 is made of a magnetic grade of stainless steel to be inductively heated by the heating induction coils 34.

A temperature sensor 38 is communicably connected to the control system 14 via the network 16 and configured to measure a current temperature of the thermal treatment vessel 12 in real time. In another embodiment, the temperature sensor 38 is configured to measure a current temperature of the solution 24 in the thermal treatment vessel 12 during the crystallization process in real time.

During the cooling process, a cooling device, such as a water sprayer 40 and/or a fan 42, is communicably connected to the control system 14 via the network 16 and configured to effectively lower the current temperature of the solution 24. In one embodiment, the cooling process is performed using only ambient or natural air associated with the thermal treatment vessel 12. In embodiments, any combinational use of the ambient air, the water sprayer 40, and the fan 42 is contemplated to suit the application.

A data storage 44 is communicably connected to the control system 14 via the network 16 and configured to store temporarily or permanently any data related to the crystallization process performed by the crystallization system 10. In embodiments, the data storage 44 can be a non-transitory computer-readable storage medium that stores one or more instructions executed by the control system 14, and the data stored in the storage 44 can be electrically retrieved by the control system 14 for further processing.

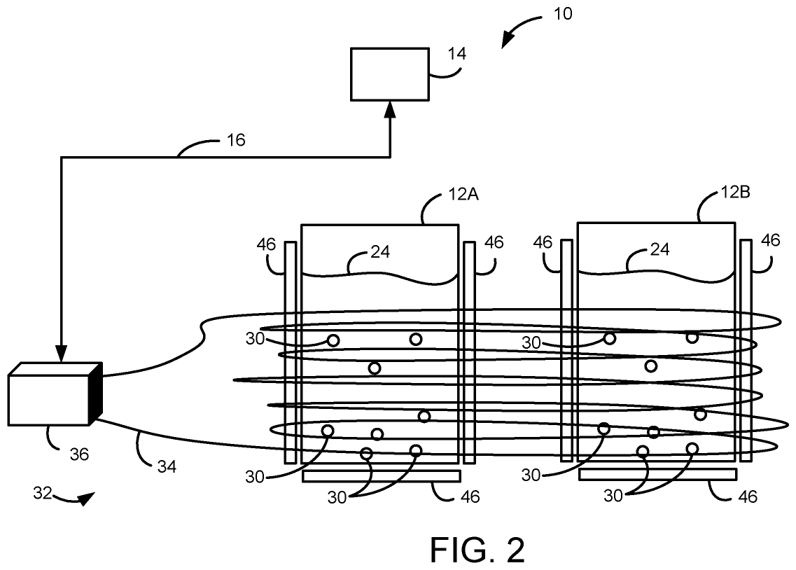

Referring now to FIG. 2, another exemplary configuration of the thermal treatment vessel 12 used in the crystallization system 10 of FIG. 1 is shown. In the illustrated embodiments of FIGS. 1 and 2, the high-frequency power supply unit 36 generates and passes a high-frequency current enough to increase the temperature of the thermal treatment vessel 12 to a predetermined treatment temperature, e.g., 270° C., for the solution 24 to be treated using the heating induction coils 34.

An exemplary energization frequency of the high-frequency power supply unit 36 can be approximately 20 kilohertz (KHz) and a maximum output can be approximately 20 kilowatts (KW). However, the frequency and the maximum output of the high-frequency power supply unit 36 can vary to suit different applications.

In one embodiment, the high-frequency power supply unit 36 is used to inductively drive the heating induction coils 34 for the predetermined treatment time period. For example, the high-frequency power supply unit 36 drives the heating induction coils 34 to increase the current temperature of the solution 24 in the thermal treatment vessel 12, 12A, 12B to the predetermined treatment temperature of 270° C. for 30 minutes. In embodiments, the predetermined treatment time period relates to a residence time of the solution 24 in the thermal treatment vessel 12 for obtaining an optimal crystallization progression.

In embodiments, one or more thermal treatment vessels 12, 12A, 12B are included in the crystallization system 10. In FIG. 2, a plurality of thermal treatment vessels 12A, 12B are arranged in series. In the illustrated embodiment, a heat transfer area provided by the heating assembly 32 is shared by both a first thermal treatment vessel 12A and a second thermal treatment vessel 12B (both 12A and 12B being collectively designated 12). However, in certain embodiments, each of the first thermal treatment vessel 12A and the second thermal treatment vessel 12B can be separately heated by its respective heating induction coils 34.

A configuration difference of the illustrated embodiment of FIG. 2 is the absence of the temperature sensor 38 (FIG. 1). In some embodiments, the temperature sensor 38 provides information about the current temperature of the thermal treatment vessel 12 and/or the solution 24 during the crystallization process. Based on the current temperature of the thermal treatment vessel 12 and/or the solution 24, the control system 14 operates the heating assembly 32. However, in FIG. 2, a magnetic shunt alloy layer 46 is included in the crystallization system 10 in lieu of the temperature sensor 38.

In one embodiment, the magnetic shunt alloy layer 46 is made of a material configured to cover at least a portion of an outer surface of the thermal treatment vessel 12, 12A, 12B. In one example, the magnetic shunt alloy layer 46 is removably connected to at least the portion of the outer surface of the thermal treatment vessel 12, 12A, 12B. In another example, the magnetic shunt alloy layer 46 is fixedly attached to at least the portion of the outer surface of the thermal treatment vessel 12, 12A, 12B.

In embodiments, the magnetic shunt alloy layer 46 has a characteristic wherein magnetism of the magnetic shunt alloy layer 46 is automatically lost when a current temperature of the magnetic shunt alloy layer 46 reaches a predetermined temperature (e.g., 270° C.) representing a magnetic transformation point. An exemplary magnetic transformation point of the magnetic shunt alloy layer 46 represents a Curie temperature above which ferromagnetic materials lose their relative magnetic permeability such that magnetism disappears above this temperature and behave paramagnetically.

An exemplary range of the Curie temperature is approximately between 100-750° C. In one embodiment, the magnetic shunt alloy layer 46 is made of a magnetic shunt alloy having a magnetic function at or below the Curie temperature of 270° C. and a non-magnetic function above the Curie temperature of 270° C. In this configuration, when the magnetic shunt alloy layer 46 is inductively heated by the heating assembly 32, one of the magnetic function and the non-magnetic function is automatically selected and performed based on the current temperature of the magnetic shunt alloy layer 46.

Thus, without using any individual heating system, when the current temperature of the magnetic shunt alloy layer 46 is greater than the predetermined temperature of 270° C., the magnetic shunt alloy layer 46 automatically loses the relative magnetic permeability and its magnetism disappears. Yet, when the current temperature of the magnetic shunt alloy layer 46 is less than or equal to the predetermined temperature of 270° C., the magnetic shunt alloy layer 46 maintains the relative magnetic permeability and its magnetism allows continuous inductive heating caused by the heating assembly 32.

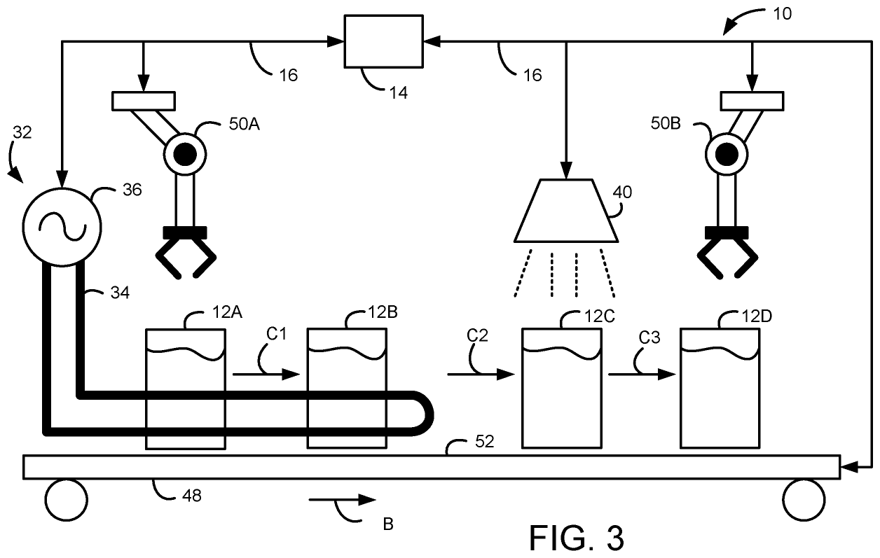

Referring now to FIG. 3, an exemplary arrangement of the thermal treatment vessels 12A, 12B, 12C, 12D is shown (12A-12D being collectively designated 12). In this arrangement, a delivery system 48, such as a conveyor device, is communicably connected to the control system 14 via the network 16 and configured to serially advance one or more thermal treatment vessels 12A, 12B, 12C, 12D in a flow direction designated by an arrow B.

In the illustrated embodiment, a first robotic system 50A is communicably connected to the control system 14 via the network 16 and configured to deliver a first thermal treatment vessel 12A to a first position of the delivery system 48. As the first thermal treatment vessel 12A is inductively heated by the heating assembly 32, the first thermal treatment vessel 12A advances to a second position of the delivery system 48 in a flow direction designated by an arrow C1 using a motion member 52, such as a conveyor belt.

In one example, a second thermal treatment vessel 12B can be the same first thermal treatment vessel 12A that advanced from the first position to the second position. In another example, the second thermal treatment vessel 12B can be a different thermal treatment vessel 12 that advanced to the second position during the heating process.

When the heating process is completed, the second thermal treatment vessel 12B advances to a third position of the delivery system 48 in a flow direction designated by an arrow C2 using the motion member 52. A third thermal treatment vessel 12C located at the third position enters into the cooling process performed by the control system 14.

During the cooling process, the third thermal treatment vessel 12C is cooled by the water sprayer 40. In this illustrated embodiment, the solvent 24 is cooled by water to initiate crystallization of the compound 18 in the solvent 24. Other cooling methods using forced or ambient air are also contemplated to suit different applications.

After the cooling process, the third thermal treatment vessel 12C advances to a fourth position of the delivery system 48 in a flow direction designated by an arrow C3 using the motion member 52. A fourth thermal treatment vessel 12D located at the fourth position is then retrieved by a second robotic system 50B that is communicably connected to the control system 14 via the network 16. Subsequently, the fourth thermal treatment vessel 12D can be stored at a proper location or delivered to a different location for further processing.

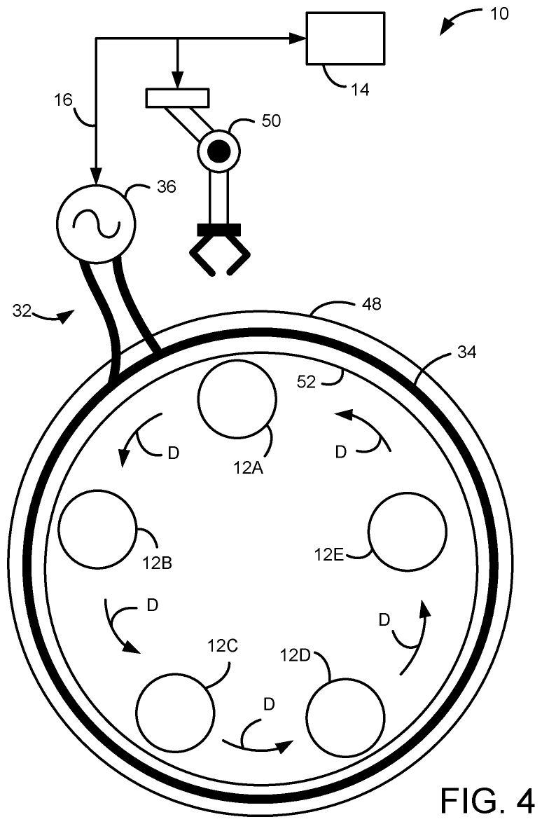

Referring now to FIG. 4, another exemplary arrangement of the thermal treatment vessels 12A, 12B, 12C, 12D, 12E is shown (12A-12E being collectively designated 12). In the illustrated embodiment, the delivery system 48 is a merry-go-round type system, configured to sequentially deliver the thermal treatment vessels 12A-12E in a flow direction designated by arrows D using the motion member 52, such as a rotating table. In this configuration, only a single robotic system 50 (e.g., 50A or 50B) is utilized for delivery and retrieval of each thermal treatment vessel 12 in relation to the motion member 52.

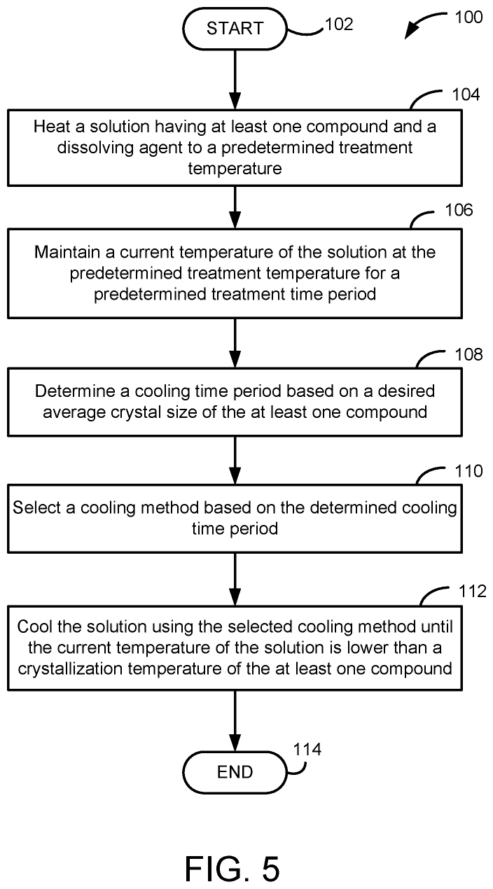

Referring now to FIG. 5, a flow chart of an exemplary method 100 of performing a crystallization process is shown for a fluidic product, such as the solution 24, having at least one compound 18, such as TPA, mixed with the dissolving agent 20 in accordance with embodiments of the present disclosure. The method 100 is shown in relation to the crystallization system 10 shown in FIGS. 1-4.

In one embodiment, the method 100 can be implemented by the control system 14 communicably connected to the heating assembly 32. In one embodiment, the steps implementing the method 100 can be in the form of computer readable program instructions stored in one of memories of electronic controllers in the control system 14 and executed by a respective processor of the electronic controllers, or other computer usable medium.

In another embodiment, the steps implementing the method 100 can be stored and executed on a module or controller, such as the control system 14, which may or may not be independent from one of the electronic controllers of the crystallization system 10. The method 100 may run continuously or may be initiated in response to one or more predetermined events, such as an initial push of a start button (not shown). Any steps of the method 100 can be executed in any order suitable for the application.

The method 100 begins in step 102. As discussed above, the crystallization process performed by the control system 14 includes the heating process and the cooling process that occurs after the heating process. In this illustrated embodiment, steps 104-106 relate to the heating process, and steps 108-112 relate to the cooling process.

In step 104, the control system 14 instructs the heating assembly 32 to inductively heat the solution 24 stored in the thermal treatment vessel 12 to a predetermined treatment temperature (e.g., 270° C.). In step 106, the control system 14 instructs the heating assembly 32 to maintain a current temperature of the solution 24 at the predetermined treatment temperature for a predetermined treatment time period (e.g., 30 minutes).

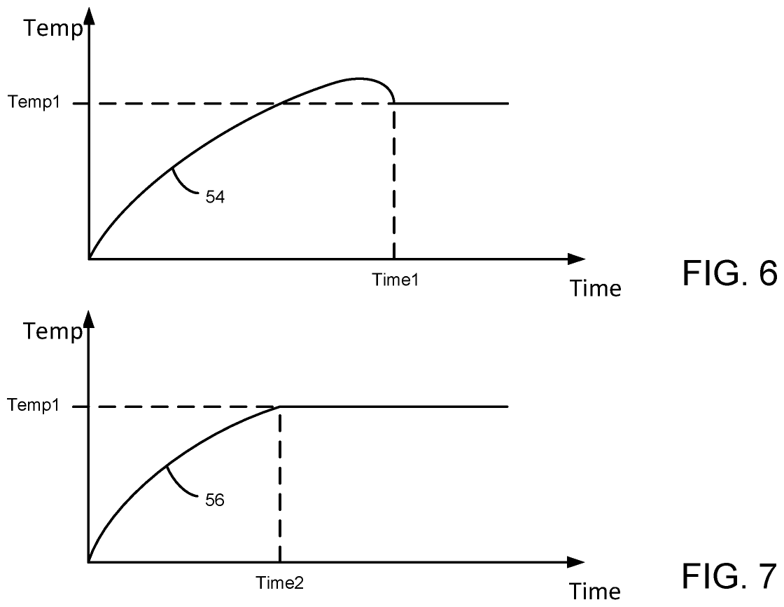

In embodiments, the predetermined treatment time period refers to a time period during which the thermal treatment vessel 12 is heated using one or more heating methods. Exemplary heating methods include induction heating, electric heating, gas heating, and the like. When the temperature sensor 38 is utilized during the heating process, the current temperature of the solution 24 can temporarily exceed the predetermined treatment temperature as shown in FIG. 6.

Referring now to FIG. 6, an exemplary graphical presentation 54 of the current temperature of the solution 24 is shown during the heating process. In FIG. 6, an X-axis represents an elapsed time Time during the heating process, and a Y-axis represents a current temperature Temp of the solution 24 during the heating process.

In FIG. 6, as the heating process progresses, the temperature Temp of the solution 24 increases due to the induction heating caused by the heating assembly 32. When the temperature Temp of the solution 24 is greater than the predetermined treatment temperature Temp1 (e.g., 270° C.), the control system 14 instructs the heating assembly 32 to cease the induction heating operation such that the temperature Temp of the solution 24 is lowered to the predetermined treatment temperature Temp1.

When the temperature Temp of the solution 24 is equal to 270° C., designated as Time1, the control system 14 instructs the heating assembly 32 to maintain the temperature Temp of the solution 24 for the predetermined treatment time period. After a lapse of the predetermined treatment time period, the heating process is considered as completed. However, as shown in FIG. 7, when the magnetic shunt alloy layer 46 is utilized during the heating process, the current temperature of the solution 24 can be controlled automatically without using the temperature sensor 38.

Referring now to FIG. 7, another exemplary graphical presentation 56 of the current temperature of the solution 24 is shown during the heating process. In FIG. 7, an X-axis represents an elapsed time Time during the heating process, and a Y-axis represents a current temperature Temp of the solution 24 during the heating process.

In FIG. 7, as the heating process progresses, the temperature Temp of the solution 24 increases due to the induction heating caused by the heating assembly 32. When the temperature Temp of the solution 24 is equal to the predetermined treatment temperature Temp1 (e.g., 270° C.), the magnetism of the magnetic shunt alloy layer 46 is automatically lost and behaves paramagnetically.

At such moment, designated as Time2, without ceasing the induction heating operation, the heating assembly 32 can maintain the temperature Temp of the solution 24 for the predetermined treatment time period. After a lapse of the predetermined treatment time period, the heating process is considered as completed. As such, an overall operation of the heating process is readily controlled without causing high operating costs and time.

Returning now to FIG. 5, the control system 14 performs the cooling process upon completion of the heating process. An important aspect of the cooling process performed by the control system 14 is that the particle size of each of the plurality of crystal particles 30 is varied based on a cooling speed of the solution 24 during the cooling process.

In one embodiment, the particle size is controlled such that a standard deviation associated with the plurality of crystal particles 30 is lower than a predetermined value, e.g., less than one (1) at a normal distribution. For example, the predetermined value of the standard deviation ranges between zero and 0.6. As such, in some embodiments, all particle sizes of crystal particles 30 can be identical with the standard deviation of zero.

In one embodiment, the cooling process defines the standard deviation such that each particle size is within one (1) standard deviation from a mean value of sizes of the plurality of crystal particles 30. As such, each particle size can be close to the mean value and thus most particle sizes are expected to be spread closely to the mean value.

Specifically, the cooling speed is determined based on a specific cooling method and/or a predetermined cooling time period in relation to the current temperature of the solution 24. In embodiments, the cooling speed determines the particle size of each of the plurality of crystal particles 30.

In embodiments, the cooling process can be performed on the thermal treatment vessel 12 by one or more cooling methods. Exemplary cooling methods include a natural cooling method, an air-cooling method, and a water-cooling method. In embodiments, the predetermined cooling time period refers to a time duration during which the cooling process lasts.

In one embodiment, the natural cooling method refers to a cooling technique wherein the thermal treatment vessel 12 is naturally cooled by ambient air (or room temperature) surrounding the thermal treatment vessel 12. In one embodiment, the air-cooling method refers to another cooling technique wherein the thermal treatment vessel 12 is cooled by forced air generated by the fan 42. In one embodiment, the water-cooling method refers to yet another cooling technique wherein the thermal treatment vessel 12 is cooled by water sprayed by the water sprayer 40.

In step 108, the control system 14 determines a predetermined cooling time period based on a desired particle size of the at least one compound 18. In certain embodiments, the desired particle size is associated with the standard deviation having a value less than 1.0. For example, at least approximately 68% (i.e., within one standard deviation of the desired particle size) of the crystal particles 30 has the desired particle size. In some embodiments, the desired particle size refers to a particle size of each of the plurality of crystal particles 30 where the standard deviation associated with the plurality of crystal particles 30 is less than 0.5.

In embodiments, the predetermined cooling time period refers to a time period during which the thermal treatment vessel 12 is cooled using one or more cooling methods. An exemplary desired particle size of TPA ranges approximately 125-200 micrometers (μm).

For example, the desired particle size of each of crystal particles of the at least one compound 18 can be received from a user or another system (e.g., via a keyboard or a touchscreen) such that most crystal particle sizes (e.g., 90%) are expected to be spread in a range of 130-150 μm. Upon receiving the desired particle size, the control system 14 reads an empirical data stored in the data storage 44 and retrieves the cooling time period associated with the desired particle size.

In embodiments, the empirical data refers to information related to the cooling time period associated with the cooling method, and a corresponding particle size of each of the plurality of crystal particles 30 associated with the compound 18. For example, using a trial-error method, the control system 14 can perform various experimental cooling operations.

During the experimental cooling operations, the empirical data can be collected that includes the cooling method, the cooling time period, and the particle size of each of the plurality of crystal particles 30 associated with the compound 18. For example, the control system 14 measures the cooling time period and performs an image analysis on each crystal particle 30 of TPA, and records the corresponding particle size of TPA in the data storage 44.

An exemplary empirical data is a look-up table representing a relationship between the cooling method, the cooling time period, and the corresponding particle size of TPA is shown in TABLE 1 below.

TABLE 1

Cooling Method

| TPA | Natural | Air | Water |
|---|---|---|---|
| Cooling Time Period | 1750 | 256 | 2 |
| Particle Size | 501-1000 | 201-500 | 125-200 |

wherein Cooling Time Period is shown in seconds and Particle Size is shown in micrometer (or micron). Determination of the cooling time period is achieved by comparison between the desired particle size and Particle Size in the empirical data. For example, Particle Size that is closest to the desired particle size is selected by the control system 14.

In step 110, the control system 14 selects a cooling method based on the determined cooling time period using the empirical data. In one embodiment, the control system 14 reads the empirical data stored in the data storage 44 and selects the cooling method associated with the determined cooling time period. For example, Cooling Method having Cooling Time Period that is closest to the determined cooling time period is selected by the control system 14.

In step 112, the control system 14 performs a cooling operation on the solution 24 stored in the thermal treatment vessel 12 using the selected cooling method until the current temperature of the solution 24 is lower than a crystallization temperature of the at least one compound 18. An exemplary crystallization temperature of TPA ranges between 160-182° C.

Referring now to FIG. 8, exemplary graphical presentations 58, 60, 62 of a relationship between the current temperature of the solution 24 and the particle size of each of the plurality of crystal particles 30 are shown during the cooling operation of the crystallization system 10. In FIG. 8, an X-axis represents a particle size Size during the cooling process, and a Y-axis represents a current temperature Temp of the solution 24 during the cooling process.

In FIG. 8, as the cooling process progresses, the temperature Temp of the solution 24 decreases due to the cooling method performed by the control system 14. A crystallization state of the solution 24 is initiated when the temperature Temp of the solution 24 is less than the crystallization temperature Temp2 (e.g., 160° C.) of the at least one compound 18. At such moment, the solution 24 begins to precipitate and the initiated crystallization state causes formation the plurality of crystal particles 30 in the solution 24.

In embodiments, the predetermined treatment temperature Temp1 is greater than the crystallization temperature Temp2. When the temperature Temp of the solution 24 decreases down to a predetermined termination temperature Temp3 (e.g., 100° C.), the cooling process is considered as completed. In embodiments, the crystallization temperature Temp2 is greater than the predetermined termination temperature Temp3.

In embodiments, the predetermined cooling time period refers to the time duration during which the temperature Temp of the solution 24 is between the predetermined treatment temperature Temp1 and the predetermined termination temperature Temp3. For example, the predetermined cooling time period is the time duration from a moment where the current temperature of the solution 24 is 270° C. after the initiation of the cooling process to a moment where the current temperature of the solution 24 is 100° C.

A curve segment 58 represents the current temperature Temp of the solution 24 in relation to the particle size Size of each of the plurality of crystal particles 30 during the cooling process performed by the control system 14 using the water-cooling method. Upon completion of the cooling process, an exemplary final particle size Size1 of each of the plurality of crystal particles 30 is approximately 125 μm.

A curve segment 60 represents the current temperature Temp of the solution 24 in relation to the particle size Size of each of the plurality of crystal particles 30 during the cooling process performed by the control system 14 using the air-cooling method. Upon completion of the cooling process, an exemplary final particle size Size2 of each of the plurality of crystal particles 30 is approximately 201 μm.

A curve segment 62 represents the current temperature Temp of the solution 24 in relation to the particle size Size of each of the plurality of crystal particles 30 during the cooling process performed by the control system 14 using the natural cooling method. Upon completion of the cooling process, an exemplary final particle size Size3 of each of the plurality of crystal particles 30 is approximately 501 μm.

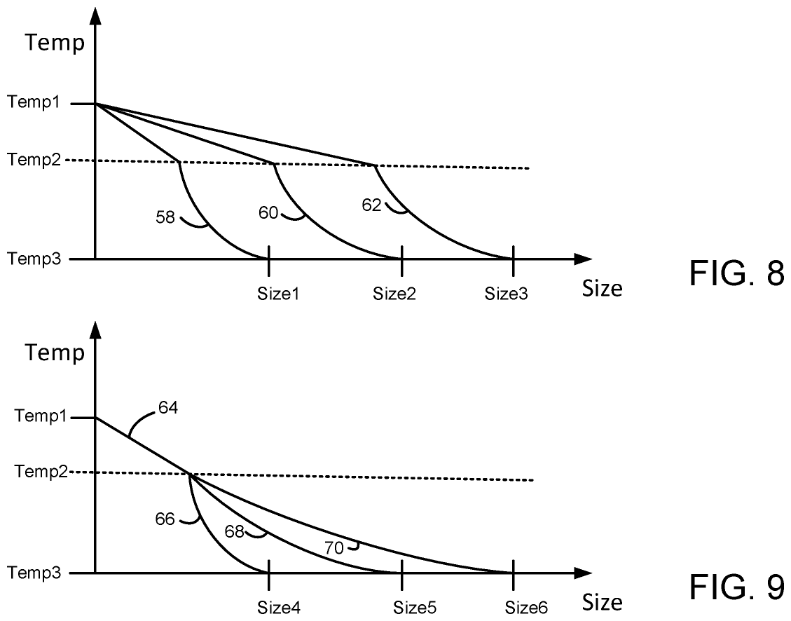

Referring now to FIG. 9, exemplary graphical presentations 64, 66, 68, 70 of a relationship between the current temperature of the solution 24 and the particle size of each of the plurality of crystal particles 30 are shown during the cooling operation of the crystallization system 10. In FIG. 9, an X-axis represents a particle size Size during the cooling process, and a Y-axis represents a current temperature Temp of the solution 24 during the cooling process.

In FIG. 9, the control system 14 utilizes at least two cooling methods during the cooling process having a plurality of cooling phases. In the illustrated embodiment, the control system 14 instructs the cooling device, such as the water sprayer 40 and/or the fan 42, to perform the air-cooling method and/or the water-cooling method during a first phase of the cooling process.

A curve segment 64 represents the first phase of the cooling process wherein the current temperature Temp of the solution 24 is decreased from the predetermined treatment temperature Temp1 (e.g., 270° C.) to the crystallization temperature Temp2 (e.g., 160° C.). When the current temperature Temp of the solution 24 is equal to the crystallization temperature Temp2, a second phase of the cooling process is performed by the control system 14.

A curve segment 66 represents the current temperature Temp of the solution 24 in relation to the particle size Size during the second phase of the cooling process performed by the control system 14 using the water-cooling method. During the second phase using the water-cooling method, the current temperature Temp of the solution 24 decreases from the crystallization temperature Temp2 to the predetermined termination temperature Temp3. Upon completion of the second phase, an exemplary final particle size Size4 of each of the plurality of crystal particles 30 is approximately 200 μm.

A curve segment 68 represents the current temperature Temp of the solution 24 in relation to the particle size Size during the second phase of the cooling process performed by the control system 14 using the air-cooling method. During the second phase using the air-cooling method, the current temperature Temp of the solution 24 decreases from the crystallization temperature Temp2 to the predetermined termination temperature Temp3. Upon completion of the second phase, an exemplary final particle size Size5 of each of the plurality of crystal particles 30 is approximately 500 μm.

A curve segment 70 represents the current temperature Temp of the solution 24 in relation to the particle size Size during the second phase of the cooling process performed by the control system 14 using the natural cooling method. During the second phase using the natural cooling method, the current temperature Temp of the solution 24 decreases from the crystallization temperature Temp2 to the predetermined termination temperature Temp3. Upon completion of the cooling process, an exemplary final particle size Size6 of each of the plurality of crystal particles 30 is approximately 1000 μm. As such, an overall operation of the cooling process is highly flexible in controlling the particle size of a final product (e.g., PTA) of the crystallization process.

Returning now to FIG. 5, the method 100 ends in step 114 and control may return to step 102. One or more of steps 102-114 can be repeated as desired.

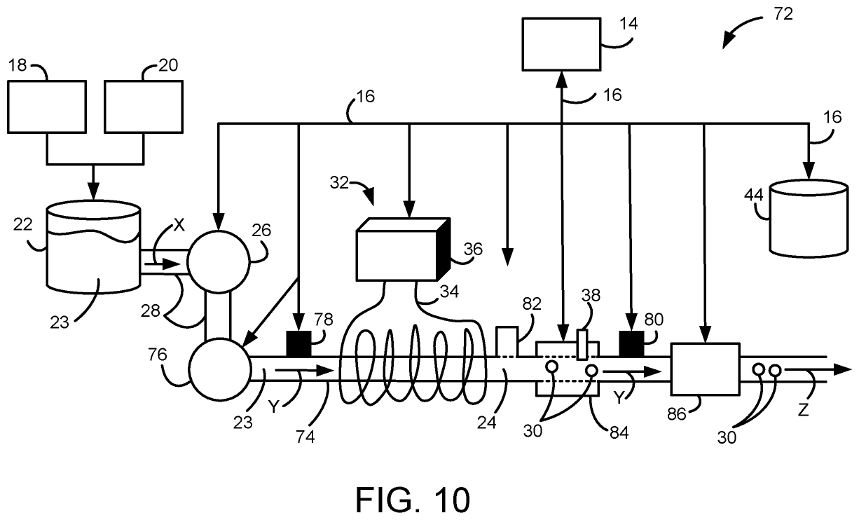

Referring now to FIG. 10, another exemplary crystallization system 72 having an inline thermal treatment vessel 74 is shown in accordance with embodiments of the present disclosure. In the illustrated embodiment, the crystallization system 72 includes the control system 14 communicably connected to various components associated with the inline thermal treatment vessel 74 via the network 16, and configured to instruct the various components for operation of the crystallization process.

An important aspect of the crystallization system 72 is that the inline thermal treatment vessel 74 is configured to perform the crystallization process by continuously delivering the sludge 23 into the inline thermal treatment vessel 74. In one embodiment, the pump 26 is used to retrieve the sludge 23 from the mixer 22 via the conduit 28 in a flow direction designated by an arrow X.

In one embodiment, the inline thermal treatment vessel 74 includes an elongated body having a tubular or cylindrical shape at a predetermined length. In the tubular shaped configuration, an inner diameter of the inline thermal treatment vessel 74 can vary to suit different applications. In one example, the inline thermal treatment vessel 74 can perform as a microreactor, micro-structured reactor, or microchannel reactor. In this configuration, the particle sizes of crystal particles 30 can be uniformized such that approximately 100% of particle sizes are identical (e.g., standard deviation of particle sizes<0.1).

Depending on characteristics of the sludge 23, a configuration of the inline thermal treatment vessel 74, such as the predetermined length, can vary to suit different applications. At one end of the inline thermal treatment vessel 74, a high-pressure pump 76 is fluidically connected to the inline thermal treatment vessel 74 and configured to continuously deliver the sludge 23 into the inline thermal treatment vessel 74 for the crystallization process.

Upon completion of the delivery of the sludge 23, the sludge 23 is continuously pushed forward in the inline thermal treatment vessel 74 in a flow direction designated by an arrow Y under the action of the high-pressure pump 76. In embodiments, the solution 23 is heated in the inline thermal treatment vessel 74 until the compound 18 is melted in the sludge 23, and at such time, the sludge 23 becomes the solution 24.

As with the crystallization system 10 described above, an exemplary treatment temperature of the crystallization system 72 ranges approximately between 100-750 degrees Celsius (° C.). An exemplary treatment time period also ranges approximately between 1-60 minutes. However, the treatment temperature range and the treatment time period range can vary to suit different applications. In one embodiment, the treatment temperature ranges approximately between 100-400° C., and in another embodiment, the treatment temperature ranges approximately between 100-300° C. For example, the treatment temperature is approximately 345° C.

In FIG. 10, during the thermal treatment induced by the heating assembly 32, the inline thermal treatment vessel 74 can reach the predetermined treatment temperature of approximately 270° C. and a predetermined inner pressure of approximately 5.5 MPa. In one example, the crystallization system 72 performs a subcritical water treatment for the crystallization process of TPA.

In one embodiment, the inline thermal treatment vessel 74 is configured for securely storing a proper amount of the solution 24 and also configured to be heated by the heating assembly 32 for functioning as the heating kiln body. In operation, the crystallization system 72 performs the crystallization process using thermal treatment for generating the plurality of crystal particles 30 associated with the compound 18.

In one embodiment, the heating assembly 32 includes one or more heating induction coils 34 configured to surround at least a portion of the inline thermal treatment vessel 74, and the high-frequency power supply unit 36 configured to inductively drive the heating induction coils 34. In one embodiment, the inline thermal treatment vessel 74 is made of a magnetic grade of stainless steel to be inductively heated by the heating induction coils 34.

A first pressure sensor 78 is disposed upstream of the heating assembly 32 and a second pressure sensor 80 is disposed downstream of the heating assembly 32. Both of the first pressure sensor 78 and the second pressure sensor 80 are configured to measure an inner pressure of the inline thermal treatment vessel 74. Other suitable locations of the first pressure sensor 78 and the second pressure sensor 80 are also contemplated to suit different applications.

In one embodiment, the first pressure sensor 78 is configured to measure the inner pressure of the inline thermal treatment vessel 74 upstream of the heating induction coils 34. Also, the second pressure sensor 80 is configured to measure the inner pressure of the inline thermal treatment vessel 74 downstream of the heating induction coils 34.

For example, both of the first pressure sensor 78 and the second pressure sensor 80 are communicably connected to the control system 14 via the network 16 to monitor the inner pressure of the inline thermal treatment vessel 74 during the crystallization process in real time. In one embodiment, the control system 14 is configured to control the high-pressure pump 76 via the network 16 based on a signal received from at least one of: the first pressure sensor 78 and the second pressure sensor 80.

A pressure relief valve 82 is configured to relieve the inner pressure of the inline thermal treatment vessel 74 and disposed downstream of the heating assembly 32. Other suitable locations of the pressure relief valve 82, such as upstream of the heating assembly 32, are also contemplated to suit different applications. In one example, the pressure relief valve 82 is an automatic compression check valve, and in another example, opening and closing operations of the pressure relief valve 82 are automatically controlled by the control system 14.

One or more temperature sensors 38 are communicably connected to the control system 14 via the network 16 and configured to measure a current temperature of the inline thermal treatment vessel 74 in real time. In another embodiment, the temperature sensor 38 is configured to measure a current temperature of the solution 24 in the inline thermal treatment vessel 74 during the crystallization process in real time.

During the cooling process, the cooling device, such as a cooling jacket 84, is communicably connected to the control system 14 via the network 16 and configured to effectively lower the current temperature of the solution 24. In one example, the cooling jacket 84 is using only ambient or natural air, and in another example, the cooling jacket 84 is using a suitable coolant or cooling agent, such as water. Any combination of the water and air is also contemplated to suit the application. Temperatures of the water and/or air can be controlled by the control system 14, e.g., using the temperature sensor 38.

In FIG. 10, for example, the cooling jacket 84 is using the water to cool the solution 24 in the inline thermal treatment vessel 74. As the solution 24 is cooled by the cooling jacket 84, one or more crystal particles 30 are formed in the inline thermal treatment vessel 74. Due to an inner pressure caused by the high-pressure pump 76, the plurality of crystal particles 30 travels in the inline thermal treatment vessel 74 in a flow direction designated by an arrow Z and is subsequently discharged for further processing.

At an opposite end of the inline thermal treatment vessel 74, the crystal particles 30 are delivered out of the inline thermal treatment vessel 74 for further processing as desired. For example, the delivered crystal particles 30 can be the final product of PTA having the desired particle size upon completion of the crystallization process.

A backpressure regulator 86 is fluidically connected to the inline thermal treatment vessel 74 and configured to maintain an upstream pressure (or backpressure) of the crystallization system 72. Various automated operations of the backpressure regulator 86 are achieved by the controller 14.

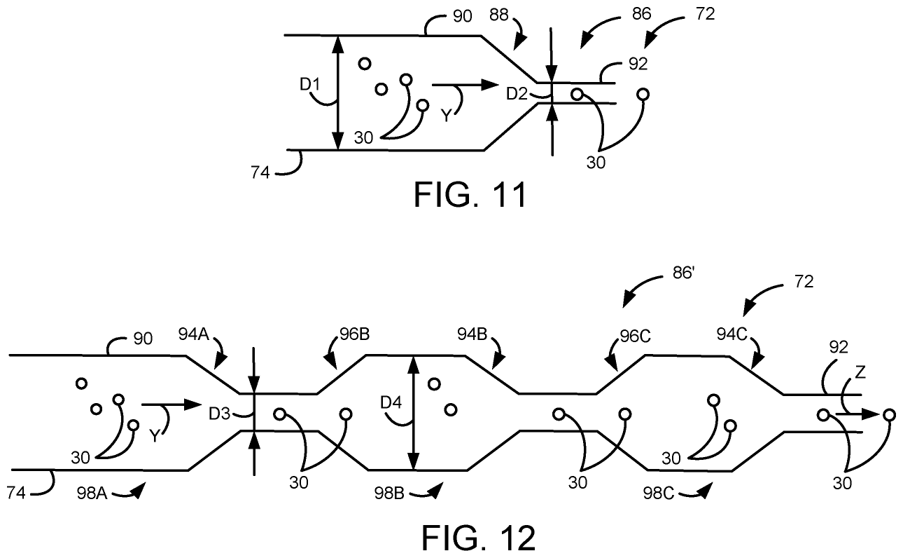

In one embodiment, the controller 14 is communicably connected to the backpressure regulator 86 via the network 16. In embodiments, the network 16 can include a wired and/or wireless data transmission interface. Detailed descriptions of the backpressure regulator 86 are provided below in paragraphs relating to FIGS. 11 and 12.

Further, the data storage 44 is communicably connected to the control system 14 via the network 16 and configured to store temporarily or permanently any data related to the crystallization process performed by the crystallization system 72. In embodiments, the data storage 44 can be a non-transitory computer-readable storage medium that stores one or more instructions executed by the control system 14, and the data stored in the storage 44 can be electrically retrieved by the control system 14 for further processing.

Referring now to FIG. 11, an exemplary backpressure regulator 86 used with the crystallization system 72 is shown. In the illustrated embodiment, the backpressure regulator 86 is fluidically connected to the inline thermal treatment vessel 74 and includes a funnel- or cone-shaped portion 88 configured to restrict a flow of the crystal particles 30 received from the inline thermal treatment vessel 74. An exemplary flow rate of the crystal particles 30 is approximately 200 milliliters per minute during operation.

In FIG. 11, the backpressure regulator 86 of the crystallization system 72 has an inlet 90 and an outlet 92, wherein the funnel-shaped portion 88 is disposed between the inlet 90 and the outlet 92. Although a single inlet 90 and a single outlet 92 are shown, any number of inlets and outlets is contemplated to suit different applications.

In the illustrated embodiment, the inlet 90 of the backpressure regulator 86 is communicating with and fluidically connected to a discharge end of the inline thermal treatment vessel 74. Typically, at the discharge end, the crystal particles 30 are discharged at a high pressure (e.g., generated by the high-pressure pump 76).

To help reduce the high pressure accumulated in the inline thermal treatment vessel 74, the funnel-shaped portion 88 is configured for restricting the flow of the crystal particles 30. In this configuration, for example, a discharge pressure of the crystal particles 30 is lowered from approximately 6 MPa to approximately 0 MPa (e.g., zero standard atmosphere). As a result, the crystal particles 30 are discharged at a lower pressure at the outlet 92.

In one embodiment, the funnel-shaped portion 88 is extending seamlessly and longitudinally at one end of the inline thermal treatment vessel 74 such that an inner diameter of the funnel-shaped portion 88 in the backpressure regulator 86 is gradually decreasing in the flow direction Y of the crystal particles 30. As shown in FIG. 11, for example, an upstream inner diameter D1 of the funnel-shaped portion 88 is greater than a downstream inner diameter D2 of the funnel-shaped portion 88 such that the funnel-shaped portion 88 has a continuously narrowing cross-sectional opening between the inlet 90 and the outlet 92.

An exemplary upstream inner diameter D1 of the funnel-shaped portion 88 is approximately 6 millimeters, and an exemplary downstream inner diameter D2 of the funnel-shaped portion 88 is approximately 0.5 millimeters. Other suitable configurations are also contemplated to suit different applications.

Referring now to FIG. 12, another exemplary backpressure regulator 86′ used with crystallization system 72 is shown. In the illustrated embodiment, the backpressure regulator 86′ includes three first funnel-shaped portions 94A, 94B, 94C and two second funnel-shaped portions 96B, 96C.

Specifically, each of the first funnel-shaped portions 94A, 94B, 94C is configured to restrict the flow of the crystal particles 30 in the backpressure regulator 86′. But, each of the second funnel-shaped portions 96B, 96C is configured to derestrict the flow of the crystal particles 30 in the backpressure regulator 86′.

In FIG. 12, the backpressure regulator 86′ of the crystallization system 72 has the inlet 90 and the outlet 92, wherein at least one first funnel-shaped portion 94A, 94B, 94C and at least one second funnel-shaped portion 96B, 96C are disposed between the inlet 90 and the outlet 92. Although three first funnel-shaped portions 94A, 94B, 94C and two second funnel-shaped portions 96B, 96C are shown, any number of first and second funnel-shaped portions is contemplated to suit different applications.

In the illustrated embodiment, the inlet 90 of the backpressure regulator 86′ is communicating with and fluidically connected to the discharge end of the inline thermal treatment vessel 74. As with the funnel-shaped portion 88 of FIG. 11, each of the first funnel-shaped portion 94A, 94B, 94C is configured for restricting the flow of the crystal particles 30 in the backpressure regulator 86′.

Unlike the first funnel-shaped portion 94A, 94B, 94C, each of the second funnel-shaped portion 96B, 96C is configured to extend seamlessly longitudinally such that an inner diameter of the second funnel-shaped portion 96B, 96C is gradually increasing in the flow direction Y of the crystal particles 30. As shown in FIG. 12, an upstream inner diameter D3 of the second funnel-shaped portion 96B is less than a downstream inner diameter D4 of the second funnel-shaped portion 96B such that the second funnel-shaped portion 96B has a continuously widening cross-sectional opening between the inlet 90 and the outlet 92.

An exemplary upstream inner diameter D3 of the second funnel-shaped portion 96B is approximately 2 millimeters, and an exemplary downstream inner diameter D4 of the second funnel-shaped portion 96B is approximately 6 millimeters. Other suitable configurations are also contemplated to suit different applications. In the illustrated embodiment, the first funnel-shaped portions 94A, 94B, 94C and the second funnel-shaped portions 96B, 96C are communicating with and fluidically connected with each other to provide a continuous flow path for the crystal particles 30.

To gradually lower the discharge pressure of the crystal particles 30 in the backpressure regulator 86′, a first chamber 98A, a second chamber 98B, and a third chamber 98C are included in the backpressure regulator 86′. In FIG. 12, the first chamber 98A includes one first funnel-shaped portion 94A, the second chamber 98B includes one first funnel-shaped portion 94B and one second funnel-shaped portion 96B, and the third chamber 98C includes one first funnel-shaped portion 94C and one second funnel-shaped portion 96C. In this example, a first pressure level of the first chamber 98A is approximately 6 MPa, a second pressure level of the second chamber 98B is approximately 4 MPa, and a third pressure level of the third chamber 98C is approximately 2 MPa.

In this illustrated embodiment, the pressurized crystal particles 30 received from the discharge end of the inline thermal treatment vessel 74 serially pass through the first, second, and third chambers 98A, 98B, 98C of the backpressure regulator 86′. As such, the crystal particles 30 are continuously delivered from the inlet 90 to the outlet 92 without interruption while reducing the inner pressure of the backpressure regulator 86′.

Consequently, as the crystal particles 30 travel in the flow direction Z, the discharge pressure of the crystal particles 30 at the outlet 92 becomes approximately 0 MPa (e.g., zero standard atmosphere). Although three chambers 98A, 98B, 98C are shown, any number of chambers is contemplated to suit different applications.

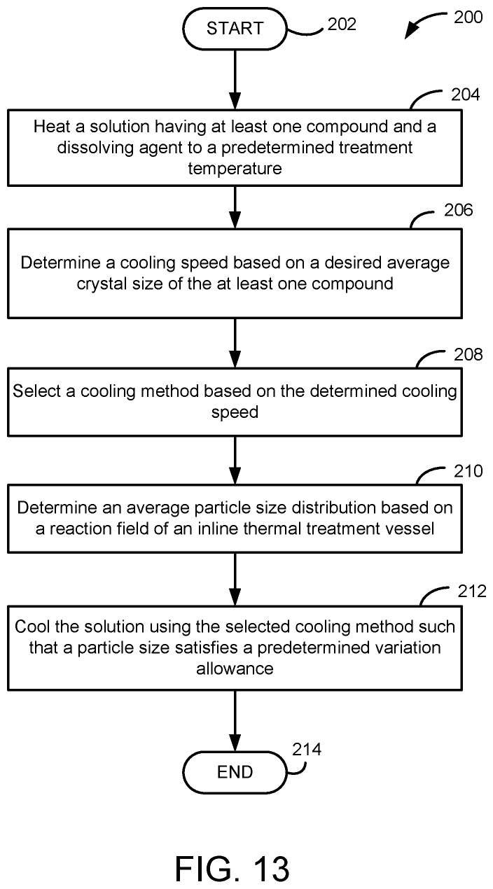

Referring now to FIG. 13, a flow chart of another exemplary method 200 of performing the crystallization process is shown for a fluidic product, such as the solution 24, having at least one compound 18, such as TPA, mixed with the dissolving agent 20 in accordance with embodiments of the present disclosure. The method 200 is shown in relation to the crystallization system 72 shown in FIGS. 10-12.

In one embodiment, the method 200 can be implemented by the control system 14 communicably connected to the heating assembly 32. In one embodiment, the steps implementing the method 200 can be in the form of computer readable program instructions stored in one of memories of electronic controllers in the control system 14 and executed by a respective processor of the electronic controllers, or other computer usable medium.

In another embodiment, the steps implementing the method 200 can be stored and executed on a module or controller, such as the control system 14, which may or may not be independent from one of the electronic controllers of the crystallization system 72. The method 200 may run continuously or may be initiated in response to one or more predetermined events, such as an initial push of a start button (not shown). Any steps of the method 200 can be executed in any order suitable for the application.

The method 200 begins in step 202. As discussed above, the crystallization process performed by the control system 14 includes the heating process and the cooling process that occurs after the heating process. In this illustrated embodiment, step 204 relates to the heating process, and steps 206-212 relate to the cooling process.

In step 204, the control system 14 instructs the heating assembly 32 to inductively heat the solution 24 stored in the inline thermal treatment vessel 74 to a predetermined treatment temperature (e.g., 270° C.). Then, the control system 14 instructs the heating assembly 32 to maintain a current temperature of the solution 24 at the predetermined treatment temperature for a predetermined treatment time period (e.g., 30 minutes).

In step 206, the control system 14 determines a cooling speed based on a desired crystal size of the at least one compound 18. For example, the desired crystal size can be a predetermined crystal size selected by a user or another system.

In embodiments, the cooling speed refers to a speed of cooling the inline thermal treatment vessel 74 using one or more of the cooling methods described above. In some embodiments, the cooling speed refers to a speed of cooling the solution 24 in the inline thermal treatment vessel 74 upon completion of the heating process.

As with the method 100, the desired crystal size of each of crystal particles of the at least one compound 18 can be received from or selected by a user or another system (e.g., via a keyboard or a touchscreen) such that most crystal particle sizes (e.g., 90% or more) are expected to be spread in a range of 130-150 μm. Upon receiving the desired particle size, the control system 14 reads an empirical data stored in the data storage 44 and retrieves the cooling speed associated with the desired particle size.

In embodiments, the empirical data further refers to information related to the cooling speed associated with the cooling method, and a corresponding particle size of each of the plurality of crystal particles 30 associated with the compound 18. For example, using a trial-error method, the control system 14 can perform various experimental cooling operations.

During the experimental cooling operations, the empirical data can be collected that includes the cooling method, the cooling speed, and the particle size of each of the plurality of crystal particles 30 associated with the compound 18. For example, the control system 14 measures the cooling speed and performs an image analysis on each crystal particle 30 of TPA, and records the corresponding particle size of TPA in the data storage 44.

An exemplary empirical data is a look-up table representing a relationship between the cooling method, the cooling speed, and the corresponding particle size of TPA is shown in TABLE 2 below.

TABLE 2

Cooling Method

| TPA | Natural | Air | Water |
|---|---|---|---|
| Cooling Speed | −0.5° C./sec | −5° C./sec | −20° C./sec |
| Particle Size | 501-1000 | 201-500 | 125-200 |

wherein Cooling Speed is shown in Celsius per second and Particle Size is shown in micrometer (or micron). Determination of the cooling speed is achieved by comparison between the desired particle size and Particle Size in the empirical data. For example, Particle Size that is closest to the desired particle size is selected by the control system 14.

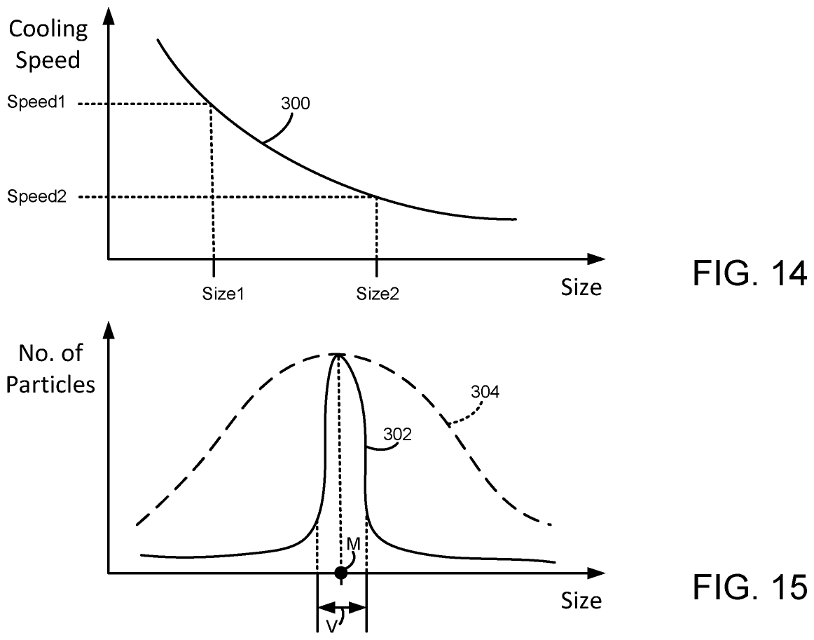

Referring now to FIG. 14, an exemplary graphical presentation of a relationship between the cooling speed and the particle size during the cooling process is shown. A curve segment 300 represents the particle size Size of each of the plurality of crystal particles 30 during the cooling process in relation to the cooling speed Cooling Speed of the solution 24 (or the inline thermal treatment vessel 74).

Upon completion of the cooling process, an exemplary final particle size Size1 of each of the plurality of crystal particles 30 is approximately 125 μm when applying a cooling speed Speed1 of −20° C. per second. In this example, when the solution 24 is cooled using the water-cooling method so that the current temperature of the solution 24 is lowered by 20 degrees Celsius per one second, the final particle size Size1 of each of the crystal particles 30 is approximately 125 μm.

In another example, the exemplary final particle size Size2 of each of the plurality of crystal particles 30 is approximately 201 μm when applying a cooling speed Speed2 of −5° C. per second. In this example, when the solution 24 is cooled using the air-cooling method so that the current temperature of the solution 24 is lowered by 5 degrees Celsius per one second, the final particle size Size2 of each of the crystal particles 30 is approximately 201 μm.

Returning now to FIG. 13, in step 208, the control system 14 selects a cooling method based on the determined cooling speed using the empirical data. In one embodiment, the control system 14 reads the empirical data stored in the data storage 44 and selects the cooling method associated with the determined cooling speed. For example, Cooling Method having Cooling Speed that is closest to the determined cooling speed is selected by the control system 14.

In step 210, the control system 14 determines a particle size based on a reaction field of the inline thermal treatment vessel 74. As similarly discussed above, the reaction field refers to the inner volume of the inline thermal treatment vessel 74. In this example, although the inline thermal treatment vessel 74 is used, other thermal treatment vessels, such as the thermal treatment vessel 12, are also contemplated to suit the application.

In one embodiment, to ensure overall conformity of the particle sizes, the particle size is determined by the control system 14 for producing the crystal particles 30 so that a majority of crystal particles 30 consistently has the desired particle size. As such, each particle size can be close to the mean value of sizes of the crystal particles 30 and thus most particle sizes are expected to be distributed as closely as possible to the mean value.

Referring now to FIG. 15, an exemplary graphical presentation of a relationship between a number of crystal particles and a particle size, designated as M, during the cooling process is shown. Each curve segment 302, 304 represents the number of crystal particles 30 produced during the cooling process in relation to the particle size M of each of crystal particles 30. In one embodiment, the particle size M refers to the desired particle size of the at least one compound 18.

More specifically, the curve segment 302 represents the number of crystal particles 30 produced by the crystallization system 72, and the curve segment 304 represents the number of crystal particles 30 produced by the existing crystallization process without using the crystallization system 72. As shown in the curve segment 304, when using conventional crystallization systems, the particle sizes of crystal particles 30 are approximately normally and widely distributed from the desired particle size M, featuring a bell-shaped distribution of the particle sizes.

However, as shown in the curve segment 302, when using the crystallization system 72, the particle sizes of the crystal particles 30 are highly concentrated around the desired particle size M. In one embodiment, a predetermined variation span, designated as V, from the desired particle size M is determined by the control system 14.

For example, the control system 14 controls the particle size having the predetermined variation span V based on the ratio R associated with the reaction field of the inline thermal treatment vessel 74. In this way, a predetermined amount (e.g., approximately 90%) of the crystal particles 30 produced by the crystallization system 72 has the desired particle size M. As such, most particle sizes are distributed as close as possible to the desired particle size M to enhance the useful physical or chemical properties of the crystal particles 30.

Returning now to FIG. 13, in step 212, the control system 14 performs a cooling operation on the solution 24 stored in the inline thermal treatment vessel 74 using the selected cooling method and the determined cooling speed until the current temperature of the solution 24 is lower than a crystallization temperature of the at least one compound 18. An exemplary crystallization temperature of TPA ranges between 160-182° C.

In one embodiment, the cooling operation is performed by the control system 14 such that each particle size of the crystal particles 30 in the solution 24 satisfies a predetermined variation allowance of the crystal particles 30 in relation to the desired crystal size M. In one example, the predetermined variation allowance refers to an acceptable variation amount from the desired particle size M (e.g., ±5 μm). In another example, the predetermined variation allowance refers to an acceptable variation percentage from the desired particle size M (e.g., ±5%).

In one embodiment, the predetermined variation allowance of the crystal particles 30 in relation to the desired crystal size M is satisfied based on the ratio R associated with the reaction field of the inline thermal treatment vessel 74 used in the cooling process. The method 200 ends in step 214 and control may return to step 202. One or more of steps 202-214 can be repeated as desired.

It should be appreciated that any steps of the method 100 and the method 200 described herein may be implemented by a process controller, or other similar component, of the control system 14. Specifically, the process controller may be configured to execute computer readable instructions for performing one or more steps of the method 100 and the method 200. In one embodiment, the process controller may also be configured to transition from an operating state, during which a larger number of operations are performed, to a sleep state, in which a limited number of operations are performed, thus further reducing quiescent power draw of an electrical power source for the crystallization system 10 and/or the crystallization system 72.

The present disclosure is more easily comprehended by reference to the specific embodiments, examples and drawings recited hereinabove which are representative of the present disclosure. It must be understood, however, that the same are provided for the purpose of illustration, and that the present disclosure may be practiced otherwise than as specifically illustrated without departing from its spirit and scope. As will be realized, the present disclosure is capable of various other embodiments and that its several components and related details are capable of various alterations, all without departing from the basic concept of the present disclosure. Accordingly, descriptions will be regarded as illustrative in nature and not as restrictive in any form whatsoever. Modifications and variations of the system, method, and apparatus described herein will be obvious to those skilled in the art. Such modifications and variations are intended to come within the scope of the appended claims.

1. A method of crystallizing a solution having at least one compound mixed with a dissolving agent, the method comprising:
performing a heating process for the solution by heating the solution until a current temperature of the solution is equal to a predetermined treatment temperature;
maintaining the current temperature of the solution at the predetermined treatment temperature for a predetermined treatment time period;
performing a cooling process for the solution by cooling the solution until the current temperature of the solution is less than the predetermined treatment temperature and a crystallization temperature of the at least one compound;
causing formation of a plurality of crystal particles of the at least one compound in the solution by cooling the solution until the current temperature of the solution is equal to a predetermined termination temperature of the at least one compound; and
varying a particle size of each of the plurality of crystal particles based on a cooling speed of the solution during the cooling process utilizing the cooling process having a plurality of cooling phases.

2. The method of claim 1, further comprising including a first phase and a second phase in the plurality of cooling phases.

3. The method of claim 2, further comprising decreasing the current temperature of the solution from the predetermined treatment temperature to the crystallization temperature of the at least one compound during the first phase.

4. The method of claim 2, further comprising decreasing the current temperature of the solution from the crystallization temperature of the at least one compound to the predetermined termination temperature of the at least one compound during the second phase.

5. The method of claim 1, further comprising storing the solution in a thermal treatment vessel having a configuration defined by a ratio of an inner volume and at least one of: an inner surface area of the thermal treatment vessel and an outer surface area of the thermal treatment vessel.

6. The method of claim 5, further comprising defining the ratio by a value of the outer surface area of the thermal treatment vessel divided by the inner volume of the thermal treatment vessel.

7. The method of claim 1, further comprising determining a cooling speed associated with crystallizing the solution based on a desired crystal size of the at least one compound.

8. The method of claim 7, further comprising selecting a cooling method used for crystallizing the solution based on the determined cooling speed.

9. The method of claim 8, further comprising performing the cooling process for the solution by cooling the solution using the selected cooling method and the determined cooling speed.

10. The method of claim 8, further comprising satisfying a predetermined variation allowance of the plurality of crystal particles of the at least one compound in the solution in relation to the desired crystal size of the at least one compound based on a ratio associated with a reaction field of a thermal treatment vessel used in the cooling process.

11. A system of crystallizing a solution having at least one compound mixed with a dissolving agent, the system comprising:
a heating assembly configured to perform a heating process for the solution by heating the solution until a current temperature of the solution is equal to a predetermined treatment temperature;
the heating assembly configured to maintain the current temperature of the solution at the predetermined treatment temperature for a predetermined treatment time period;
a cooling device configured to perform a cooling process for the solution by cooling the solution until the current temperature of the solution is less than the predetermined treatment temperature and a crystallization temperature of the at least one compound; and
a controller configured to:
cause formation of a plurality of crystal particles of the at least one compound in the solution by cooling the solution until the current temperature of the solution is equal to a predetermined termination temperature of the at least one compound; and
vary a particle size of each of the plurality of crystal particles based on a cooling speed of the solution during the cooling process utilizing the cooling process having a plurality of cooling phases.

12. The system of claim 11, wherein the plurality of cooling phases includes a first phase and a second phase.

13. The system of claim 12, wherein the controller is configured to decrease the current temperature of the solution from the predetermined treatment temperature to the crystallization temperature of the at least one compound during the first phase.

14. The system of claim 12, wherein the controller is configured to decrease the current temperature of the solution from the crystallization temperature of the at least one compound to the predetermined termination temperature of the at least one compound during the second phase.

15. The system of claim 11, further comprising a thermal treatment vessel configured to store the solution in the thermal treatment vessel having a configuration defined by a ratio of an inner volume and at least one of: an inner surface area of the thermal treatment vessel and an outer surface area of the thermal treatment vessel.

16. The system of claim 15, wherein the ratio is defined by a value of the outer surface area of the thermal treatment vessel divided by the inner volume of the thermal treatment vessel.

17. The system of claim 11, wherein the controller is configured to determine a cooling speed associated with crystallizing the solution based on a desired crystal size of the at least one compound.

18. The system of claim 17, wherein the controller is configured to select a cooling method used for crystallizing the solution based on the determined cooling speed.

19. The system of claim 18, wherein the controller is configured to perform the cooling process for the solution by cooling the solution using the selected cooling method and the determined cooling speed.

20. The system of claim 18, wherein the controller is configured to satisfy a predetermined variation allowance of the plurality of crystal particles of the at least one compound in the solution in relation to the desired crystal size of the at least one compound based on a ratio associated with a reaction field of a thermal treatment vessel used in the cooling process.
